(19) United States
(12) Patent Application Publication
(10) Pub. No.: US 2026/0287690 A1
(43) Pub. Date: Sep. 24, 2026

(54) SYSTEMS AND METHODS FOR IMPROVING A QUALITY OF A VIDEO STREAM FROM AN IMAGING BAY AND FACILITATE A REMOTE EXPERT USER TO VISUALLY GUIDE A LOCAL OPERATOR IN IMAGE ACQUISITION WORKFLOW

(71) Applicant: KONINKLIJKE PHILIPS N.V., EINDHOVEN (NL)

(72) Inventors: SIVA CHAITANYA CHADUVULA, MALDEN, MA (US); THOMAS ERIK AMTHOR, HAMBURG (DE); XINYU WANG, HAMBURG (DE); OLGA STAROBINETS, NEWTON, MA (US); FALK UHLEMANN, NORDERSTEDT (DE); EKIN KOKER, CAMBRIDGE, MA (US); RANJITH NAVEEN TELLIS, TEWKSBURY, MA (US); CHRISTIAN FINDEKLEE, NORDERSTEDT (DE); KALKANLI BEYZA, CAMBRIDGE, MA (US); JESSICA WANG, CAMBRIDGE, MA (US)

(21) Appl. No.: 19/168,350

(22) PCT Filed: Mar. 28, 2024
(86) PCT No.: PCT/EP2024/058582

§ 371 Date: Sep. 24, 2025

Related U.S. Application Data
(60) Provisional application No. 63/455,641, filed on Mar. 30, 2023.

(51) Int. Cl. G01R 33/28; G06T 5/77; G06T 7/00; G06T 7/13; G06T 11/60; H04N 7/18; H04N 23/60; H04N 23/66; H04N 23/695; H04N 23/74

(52) U.S. Cl. CPC G01R 33/283; G06T 5/77; G06T 7/0012; G06T 7/13; G06T 11/60; H04N 7/183; H04N 23/64; H04N 23/66; H04N 23/695; H04N 23/74; G06T 2200/24; G06T 2207/10016; G06T 2207/10088; G06T 2207/20081; G06T 2207/30168; G06T 2207/30232

(57) ABSTRACT

In a method for assisting a medical imaging examination of a patient, a video stream of the medical imaging examination is received. One or more artifacts in the received video stream are detected. The one or more detected artifacts in the video stream are suppressed to generate a processed video stream. The processed video stream is transmitted to an electronic processing device for use by a remote user, and displayed on a display device of the electronic processing device. At least one annotation of a base image in the video stream may be received from the remote user, and the at least one annotation superimposed on the video stream to generate an annotated video stream which is transmitted and displayed at a local operator electronic processing device operable by a local operator performing the medical imaging examination.

The following relates generally to the medical imaging arts, remote imaging assistance arts, and related arts.

### BACKGROUND

A remote operations command center (ROCC) is an infrastructure by which a remote expert (e.g. a technologist) can observe activities in an imaging bay, so as to provide advice or other assistance to a local operator (e.g., a technologist, technician, or generally a remote user) performing a medical imaging examination. Usually, the ROCC also provides mirroring of the controller display at a workstation used by the remote expert by suitable hardware and/or software such as screen mirroring software, a video stream splitter, or the like. Visibility into an imaging bay (also referred to herein as a scanner room) is key for the remote expert to support the local operator. In typical remote imaging examination monitoring systems, a camera mounted in the control room is recording video of the imaging bay through a glass. This advantageously allows remote imaging examination monitoring systems to be compatible for both magnetic resonance (MR) and/or computed tomography (CT) scanners and/or similar medical diagnostic/therapeutic equipment.

However, this kind of setup to capture video stream from the imaging bay is prone to reflections or glare from multiple light sources in the scanner/control room which results in poor visibility of the imaging bay. In addition to this, the operating conditions of the control room are often adjusted for various reasons including preserving patient privacy, which in turn, makes it hard for a remote expert to understand the events in the imaging bay based on the (low quality) video stream.

A lack of video stream quality can hinder the ability of the remote expert to assist in the examination and inhibit the remote user's efforts to orchestrate the image acquisition workflow smoothly. For example, a remote user needs accurate real-time visuals on patient and coil positioning to guide a local operator. Apart from the remote expert, AI algorithms for monitoring activity in the imaging bay also need good visibility of this real-time live video stream.

In certain implementations of remote imaging examination monitoring systems, the remote expert is responsible for running the scanner and patient's well-being, especially when local operators are present on-site. In such cases, there are specific regions in the imaging bay that the remote expert needs video with especially good visual quality. For example, if a patient is following breath commands during breath hold scans, the remote users need to watch for patient compliance. This would allow the remote user to promptly terminate a sequence before it is completed and start over if patients are uncooperative instead of waiting for the sequence to finish and having to re-do the sequence entirely due to poor image quality from breathing artifacts.

As the bay camera is typically mounted away from the glass looking into the imaging bay, there are multiple light sources in the control room. Due to this arrangement, reflections, shadows of the objects in the control room, and/or other visual artifacts can be superimposed onto the glass and eventually onto the image frames of the video stream acquired by the camera. Such interference makes it difficult for the remote expert to observe patient motion, judge patient positioning, etc. It also affects the performance of the deep learning-based techniques applied to the video stream, which is being used to further improve the user experience.

For patient safety reasons, scanners are often equipped with multiple high-intensity light sources in the imaging bay. When these light sources are within the camera's field of view, they introduce glare, reduced contrast and thereby reduce quality of the video stream, potentially creating visual discomfort for the remote expert, or hindering the ability of the remote expert to discern content of the video stream.

Unlike CT, in MR imaging bays, a radiofrequency (RF) shielding mesh located behind the glass window often contributes to a noisy image. This shield cannot be removed as it eliminates RF interference between the MR scanner and its surroundings.

Apart from these artifacts, there could be obstructions in the field of view reducing visibility into the imaging bay. For example, the technologist must restrict the camera's video stream when the exam requires the patient to be in various stages of undressing to preserve patient privacy. Such obstructions need to be detected so that certain features such as idle time calculation or inferences on the workflow step can be put on hold until the exam is completed. Sometimes, unintended obstructions such as an IV stand in the field of view of the camera can limit the remote expert's view of the imaging bay. In such cases, the local operator needs to be alerted to remove the obstruction from the camera's field of view.

Moreover, virtual guidance from the remote expert facilitates the local operator to efficiently execute image acquisition workflow steps in the imaging bay. In some remote assistance designs, local operators can verbally communicate only when they are in the control room but not in the imaging bay. This is because in these designs there is no communication channel with the expert user when the local operator is in the imaging bay. Such limited verbal communication is prone to misinterpretations and create unnecessary delays in image acquisition.

Every step in the image acquisition workflow such as patient positioning and setup of coils needs to be done correctly to avoid delays and scan repetitions. For instance, in one illustrative MR workflow, a 3-minute delay occurs each time a patient needs to be repositioned, a coil needs to be swapped, etc. Ensuring that the local user is adhering to the best practices of image acquisition workflow is particularly important for achieving operational excellence in ROCC. The remote expert can communicate such best practices to the local staff only verbally and only while they are in the control room. This poses a high cognitive load on local users to understand all the practices stated by the remote expert and implement them in the imaging bay to avoid delays.

This gets further complicated in scenarios where the remote expert is supporting a local operator who is a tech aid (that is, a local operator typically less skilled compared to regular technologist) instead of a local operator who is a technologist. In these situations, the remote expert is responsible for scanning the patient and needs confidence on whether the right body part, correct side of the patient, correct coil, and/or hearing protection are being used for the patient's exam in addition to positioning the patient appropriately.

The following discloses certain improvements to overcome these problems and others.

### SUMMARY

In one aspect, a non-transitory computer readable medium stores instructions executable by at least one electronic processor to perform a method for assisting a medical imaging examination of a patient. The method includes: receiving a video stream of the medical imaging examination; detecting one or more artifacts in the received video stream; suppressing the one or more detected artifacts in the video stream to generate a processed video stream; and transmitting the processed video stream to an electronic processing device for use by a remote user and displaying the processed video stream on a display device of the electronic processing device.

In another aspect, a system is disclosed for assisting a medical imaging examination of a patient. The system includes: a camera arranged to acquire a video stream of the medical imaging examination; a remote electronic processing device operable by a remote user monitoring the medical imaging examination; a local operator electronic processing device operable by a local operator performing the medical imaging examination and having a display device; and at least one electronic processor programmed to perform a method for assisting the medical imaging examination of a patient. The method includes: receiving the video stream of the medical imaging examination at the remote electronic processing device; receiving, via at least one input provided by the remote user on the remote electronic processing device, at least one annotation on a base image in the video stream; superimposing the at least one annotation on the video stream to generate an annotated video stream; and transmitting the annotated video stream to the local operator electronic processing device and displaying the annotated video stream on the display device of the local operator electronic processing device.

In another aspect, a method is disclosed for assisting a medical imaging examination of a patient. The method includes: monitoring the medical imaging examination by recording a video stream of the medical imaging examination using a camera located in an adjoining room next to an imaging bay within which the medical imaging examination is performed and transmitting the video stream to an electronic processing device for use by a remote user and displaying the video stream on a display device of the electronic processing device; and during the monitoring, detecting an artifact in the recorded video stream and automatically adjusting at least one of (i) the camera and/or (ii) lighting in the imaging bay and/or the adjoining room to suppress the detected artifact.

One advantage resides in removing reflections or glare in a video stream of an imaging bay transmitted to a remote monitoring room.

Another advantage resides in improving the video stream quality of an imaging bay with minimal or no-intervention from a local operator performing a medical imaging examination.

Another advantage resides in removing glare and noise in a video stream of an imaging bay transmitted to a remote monitoring room.

Another advantage resides in removing obstructions in a video stream of an imaging bay transmitted to a remote monitoring room.

Another advantage resides in providing a palette of annotations for a remote expert monitoring an imaging examination to annotate a video of the imaging examination for visualization by a local operator performing the imaging examination.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 diagrammatically shows an illustrative system for performing an examination monitoring method in accordance with the present disclosure.

FIG. 2 diagrammatically shows a cross-sectional view of an imaging bay and an adjacent control room, along with certain edge devices that are automatically controllable by the illustrative system of FIG. 1.

FIG. 3 shows example flow charts of operations suitably performed by the system of FIG. 1.

FIG. 4 shows another embodiment of the system of FIG. 1.

### DETAILED DESCRIPTION

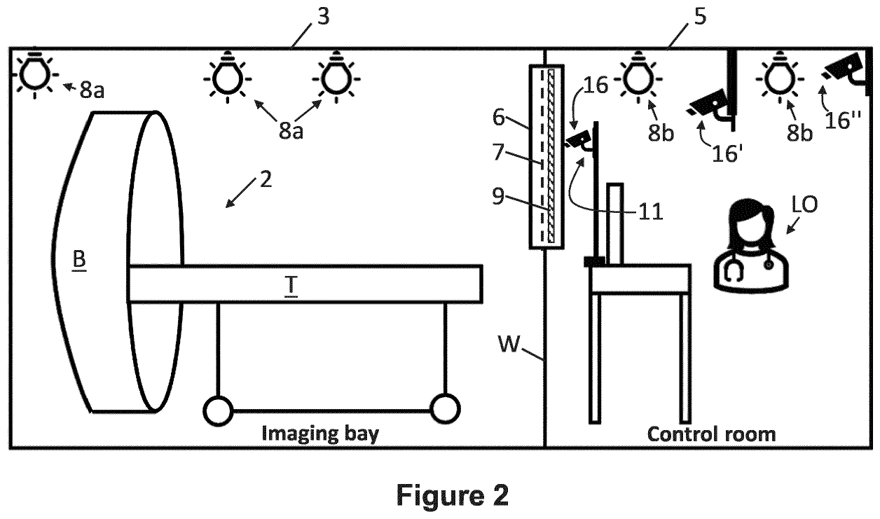

In some embodiments disclosed herein, a remote expert system is disclosed that improves a quality of a video stream from an imaging bay where an imaging procedure is occurring. With initial reference to FIG. 2, a medical imaging device 2 (for example, a CT scanner, an MR scanner, a positron emission tomography (PET) scanner, or so forth) is disposed in an imaging bay 3, and an adjoining control room 5 is also shown. The illustrative medical imaging device 2 includes a bore B into which a patient is loaded from a patient table T. FIG. 2 also illustrates a divider wall W separating the imaging bay 3 and the control room 5, and a window 6 enabling a local operator in the control room 5 to have a view of the adjacent imaging bay 3. If the medical imaging device 2 is an MR scanner, then the window 6 will typically include a radio frequency (RF) screen 7 which is part of an overall Faraday cage enclosing the imaging bay 3 to provide RF isolation of the MR scanner. The imaging bay 3 includes lighting 8a, such as LED lights, and likewise the control room 5 includes lighting 8b. During an imaging examination, a local operator LO (e.g. an imaging technologist or the like) conducts the imaging examination. FIG. 2 diagrammatically shows the local operator LO in the control room 5, but in practice the local imaging operator LO moves between the control room 5 and the imaging bay 3 as needed, e.g. going to the imaging bay 3 to assist the patient into the medical imaging device 2 and installing ancillary equipment such as local MR coils as called for in a given imaging examination, then going to the control room 5 to remotely control the medical imaging device 2 from the control room 5 to conduct imaging scans or so forth.

With continuing reference to FIG. 2, an imaging bay camera 16 is typically located in the adjoining control room 5 and positioned to view the imaging bay 3 through the window 6 between the bay and control rooms. FIG. 2 illustrates a bay camera 16 mounted close to the window 6, but also illustrates the bay camera in alternative positions, such as an illustrative ceiling-mounted bay camera 16′ and an illustrative wall-mounted bay camera 16″. The window 6 can cause reflections that can degrade the quality of video of the imaging bay 3 captured by the bay camera 16, and the RF screen 7 (in the case of MR imaging) can introduce noise from the RF screen 7 to the video. Glare can be present due to the bright lights 8a in the imaging bay 3, and/or the bright lights 8b in the control room 5. These imaging artifacts can degrade the quality of the imaging bay video captured by the bay camera 16, which in turn can negatively impact the ability of the remote expert to observe what is going on in the imaging bay 3 via the bay video.

The disclosed system provides various approaches for processing the video stream of the bay camera 16 to suppress or remove such defects. An artifact detection module detects instances of artifacts using machine learning/deep learning (ML/DL) analysis or traditional computer vision algorithms. Suitable image processing is then applied to suppress detected artifacts. For example, an edge detector can be applied to detect a regular grid artifact corresponding to the Faraday cage metal mesh, and image inpainting by dilation or other suitable processing can be applied to reduce or remove this artifact. After artifact suppression, a video quality assessment can be performed on the raw and processed video to determine which video stream is best and send that to the remote expert. Additionally, or alternatively, the video quality assessment of the processed video can be used as feedback to adjust the process, for example, if the assessment indicates overprocessing, then the amount of processing can be reduced. In some embodiments, human feedback from the remote operator can be acquired to provide training data for evaluation and training the ML algorithms used in the analysis.

In another aspect, the video analysis can perform object recognition to identify objects in the bay camera video, and if an important object is subsequently blocked by the local operator, the patient, window blinds, or the like, then an alert to the local operator and a note to that effect can be superimposed on the video sent to the remote expert.

With reference again to FIG. 2, in another aspect, “edge devices” such as the bay and/or control room lighting 8a and/or 8b can be remotely operable so that the system can automatically adjust the lighting to reduce glare or other imaging artifacts. In another example, the window 6 can include remotely operable blinds 9. As yet another example of an edge device, the bay camera 16 can include a pan/tilt/zoom (PTZ) control 11 that can be automatically controlled on the basis of the bay video analysis to improve the image quality. For example, if the control room lighting is producing glare it can be automatically dimmed.

The disclosed system is also configured to facilitate a remote expert to visually guide the local operator in image acquisition workflow. This addresses difficulties experienced by the remote expert in conveying instructions to the local operator. Typically, this is done verbally or in some cases using hand gestures in the case of a video call. It can be difficult to convey complex instructions verbally or by hand gestures. As recognized herein, the bay video can be advantageously annotated to convey such information graphically.

In some embodiments, a current frame of the bay video acquired by the bay camera 16 is presented to the remote expert as a base image, and the remote expert can draw on or otherwise annotate the base image to illustrate what he or she wants to convey to the local operator. For example, the patient, a local RF coil, or other components can be illustrated in their correct positions.

Since the remote expert may not be an artist, the illustrative embodiment provides a palette of graphical objects representing the patient and various components of the imaging system (e.g. the local RF coil), and a drag-and-drop graphical user interface (GUI) is provided by which the remote expert can select a graphical object from the palette and place it into correct position on the base image, including suitable rotation, resizing, and so forth done using handles on the graphical object.

Implementation suitably includes providing a window showing the base image and the drag-and-drop GUI on a tablet computer, workstation display, or the like operated by the remote expert, and a display in the imaging bay that is viewable by the local operator.

In a variant embodiment, a three-dimensional (3D) rendering of the imaging bay is constructed based on the bay video, and the GUI provides this 3D rendering with a palette of 3D graphical objects.

In another aspect, a current inventory of available components for use in imaging procedures performed in the imaging bay is maintained. If a component is inoperative or missing, then the corresponding graphical object may be labeled accordingly in the palette and the remote expert will be unable to drag-and-drop that unavailable component. The current inventory may be generated in several ways, such as by analysis of the bay video to detect components in view, accessing an electronic inventory of the radiology department, utilizing a real-time locating service (RTLS) if the components have RTLS tags, or so forth. Optionally, the remote expert may be able to update the inventory manually, e.g., if the local operator informs the remote expert a given MR coil is not working, the remote expert can then manually mark the corresponding palette graphical object accordingly.

In another aspect, a checklist of items can be superimposed on the base image or shown in a separate window of the GUI. As items are completed, checkboxes or the like associated with items of the checklist are manually checked off. In a variant embodiment, items can be automatically checked off if analysis of the bay video determines an item is complete. For example, if a checklist item is positioning the patient on the imaging table in a prone position, video analysis can detect when the patient is in this position and the corresponding checkbox automatically checked off. (Similarly, if the patient moves out of that position, this change can be automatically detected and the item unchecked, possibly along with a warning that the previously checked item is no longer complete).

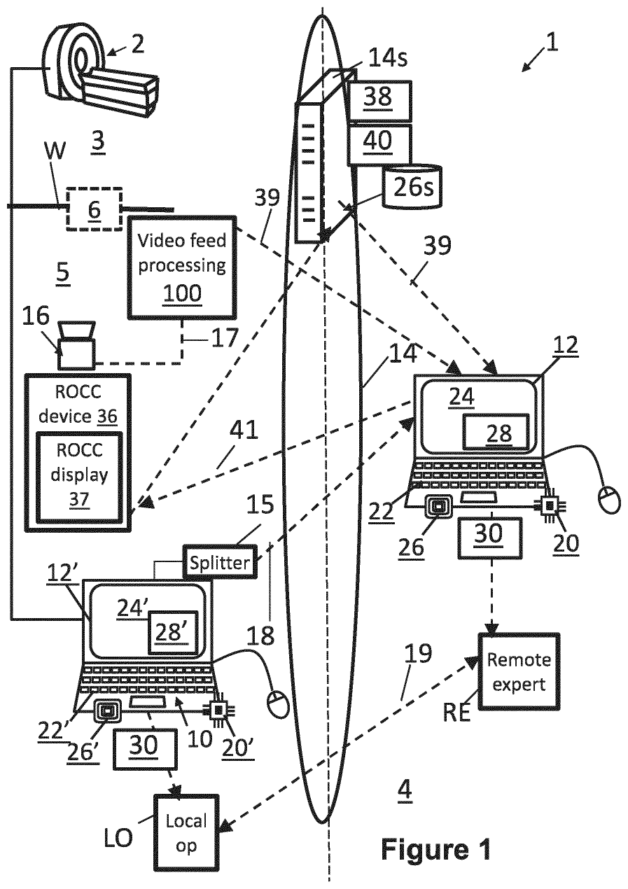

With reference to FIG. 1 and continuing reference to FIG. 2, a system 1 for providing assistance from a remote expert RE (i.e., an experienced imaging technician or a radiologist) to the local operator LO (for example, an imaging technologist or tech aid) is shown. Such a system 1 is also referred to herein as a radiology operations command center (ROCC 1). As shown in FIG. 1, the medical imaging device (also referred to as an image acquisition device, imaging device, and so forth) 2 is located in the medical imaging device bay 3 as shown in FIG. 2, while the remote expert RE is disposed in a remote location or center (hereinafter remote location) 4, and the local operator LO operates a medical imaging device controller 10 in the control room 5 as shown in FIG. 2. It should be noted that the remote expert RE may not necessarily directly operate the medical imaging device 2, but rather provides assistance to the local operator LO in the form of advice, guidance, instructions, or the like. Furthermore, in embodiments in which no ROCC is being used, the local operator LO simply corresponds to an imaging device operator or technologist (i.e., there is no remote expert in these embodiments).

The medical imaging device 2 can be a Magnetic Resonance (MR) image acquisition device, a Computed Tomography (CT) image acquisition device; a positron emission tomography (PET) image acquisition device; a single photon emission computed tomography (SPECT) image acquisition device; an X-ray image acquisition device; an ultrasound (US) image acquisition device; or a medical imaging device of another modality. The medical imaging device 2 may also be a hybrid imaging device such as a PET/CT or SPECT/CT imaging system. While a single medical imaging device 2 is shown by way of illustration in FIG. 1, more typically a medical imaging laboratory will have multiple image acquisition devices, which may be of the same and/or different imaging modalities, and the discussion here focuses on a single imaging bay 3 and a single corresponding control room 5. Moreover, the remote service center 4 may provide service to multiple hospitals. The local operator LO controls the medical imaging device 2 via an imaging device controller 10 in the control room 5. The remote expert RE is stationed at a remote workstation or electronic processing device 12 (or, more generally, an electronic controller 12 or an electronic processing device 12) which is observable by the remote expert RE. The electronic processing device 12 is used by the remote expert RE, who may also be referred to as a remote user RE.

The imaging device controller 10 includes an electronic processor 20′, at least one user input device such as a mouse 22′, a keyboard, and/or so forth, and a display device 24′. The imaging device controller 10 presents a device controller graphical user interface (GUI) 28′ on the display 24′ of the imaging device controller 10, via which the local operator LO accesses device controller GUI screens for entering the imaging examination information such as the name of the local operator LO, the name of the patient and other relevant patient information (e.g. gender, age, etc.) and for controlling the (typically robotic) patient support to load the patient into the scanner bore B (see FIG. 2) or other imaging examination region of the medical imaging device 2, selecting and configuring the imaging sequence(s) to be performed, acquiring preview scans to verify positioning of the patient, executing the selected and configured imaging sequences to acquire clinical images, displaying the acquired clinical images for review, and ultimately storing the final clinical images to a Picture Archiving and Communication System (PACS) or other imaging examinations database. In addition, the remote service center 4 (and more particularly the remote workstation 12), and the medical imaging device controller 10 in the control room 5 are in communication with each other via a communication link 14, which typically comprises the Internet augmented by local area networks at the remote operator RE and local operator LO ends for electronic data communications.

As diagrammatically shown in FIG. 1 and shown in further detail in FIG. 2, the bay camera 16 (e.g., a video camera) is arranged to acquire a video stream 17 of a portion of the medical imaging device bay 3 that includes at least the area of the medical imaging device 2 where the local operator LO interacts with the patient, and optionally may further include the imaging device controller 10. The video stream 17 is a raw video stream acquired by the bay camera 16, and can include glare or other artifacts. The video stream 17 is processed by video feed processing 100 to remove the glare and/or other artifacts so as to produce a processed video feed 39 that is sent to the remote workstation 12 via the communication link 14, e.g., as a streaming video feed received via a secure Internet link. As disclosed herein, the video stream processing 100 operates to improve the video quality by operations such as removing glare, noise, and/or so forth, and/or by controlling edge devices such as the lighting 8a, 8b to reduce such artifacts in the video stream 17. As shown in FIG. 2, the bay camera that acquires the video feed or stream 17 can be a bay camera 16 positioned on a desk where the controller 10 is disposed, or a bay camera 16′ mounted to a ceiling of the control room 5, or a bay camera 16″ mounted to a wall of the control room 5, or any combination of a number of cameras thereof. For simplicity, the bay camera 16 is primarily referred to herein. The bay camera 16 is positioned to have a field of view through the window 6 to obtain the examination video stream 17 of the imaging examination performed in the medical imaging device bay 3 using the medical imaging device 2.

Furthermore, a controller display video stream 18 is provided to enable display of a mirror view of the display device 24′ of the medical imaging device controller 10 at the remote workstation 12. The controller display video stream 18 may be provided by a video cable connecting an auxiliary video output (e.g. aux vid out) port of the imaging device controller 10 to the remote workstation 12 used by the remote expert RE. Alternatively, an illustrative video cable splitter 15 may split the controller display video stream 18 that is supplied to the remote workstation 12 via the Internet 14. Alternatively, the controller display video stream 18 may be generated by screen sharing software running on the imaging device controller 10 which captures a real-time copy of the display 24′ of the imaging device controller 10, and this copy is sent as the controller display video stream 18 from the imaging device controller 10 to the remote workstation 12. These are merely nonlimiting illustrative examples.

Hence, the remote expert RE based at the remote workstation 12 is given situational awareness of activities in the imaging bay 3 at least via the processed bay video stream 39 from the bay camera 16 and the controller display video stream 18 which provides a live real-time copy of the controller display being presented in the adjacent control room 5. Optionally, an audio feed may also be provided by a bay microphone (not shown). The communication link 14 also provides a natural language communication pathway 19 for verbal and/or textual communication between the local operator LO and the remote expert RE, in order to enable the latter to assist the former in performing the imaging examination. For example, the natural language communication link 19 may be a Voice-Over-Internet-Protocol (VOIP) telephonic connection, a videoconferencing service, an online video chat link, a computerized instant messaging service, or so forth. Alternatively, the natural language communication pathway 19 may be provided by a dedicated communication link that is separate from the communication link 14 providing the data communications 39, 18, e.g., the natural language communication pathway 19 may be provided via a landline telephone. In addition, the natural language communication pathway 19 can also be established between the remote expert RE in the remote service center 4, and the patient in the medical imaging device bay 3, thus allowing direct communication between the remote expert RE and the patient. These are again merely nonlimiting illustrative examples.

FIG. 1 also shows the remote service center 4 including the remote electronic processing device 12, such as the illustrative workstation computer 12, or more generally a computer, which is operatively connected to receive and present the processed video stream 39 of the medical imaging device bay 3 from the camera 16 and to present the controller display video stream 18 as a mirrored screen. Additionally, or alternatively, the remote electronic processing device 12 can be embodied as a server computer or a plurality of server computers 14s, e.g., interconnected to form a server cluster, cloud computing resource, or so forth. The remote electronic processing device 12 includes typical components, such as an electronic processor 20 (e.g., a microprocessor), at least one user input device (e.g., a mouse, a keyboard, a trackball, and/or the like) 22, and at least one display device 24 (e.g., an LCD display, plasma display, cathode ray tube display, and/or so forth). In some embodiments, the display device 24 can be a separate component from the workstation 12. The electronic processor 20 is operatively connected with one or more non-transitory storage media 26. The non-transitory storage media 26 may, by way of nonlimiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth; and may be for example a network storage, an internal hard drive of the remote electronic processing device 12, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include generating a graphical user interface (GUI) 28 for display on the remote operator display device 24.

The medical imaging device controller 10 in the control room 5 also includes similar components as the remote electronic processing device 12 disposed in the remote service center 4. Except as otherwise indicated herein, features of the medical imaging device controller 10 disposed in the control room 5 similar to those of the remote electronic processing device 12 disposed in the remote service center 4 have a common reference number followed by a “prime” symbol (e.g., processor 20′, display 24′, GUI 28′) as already described. In particular, the medical imaging device controller 10 is configured to display the imaging device controller GUI 28′ on a display device or controller display 24′ that presents information pertaining to the control of the medical imaging device 2 as already described, such as imaging acquisition monitoring information, presentation of acquired medical images, and so forth. The real-time copy of the display 24′ of the controller 10 (i.e., the controller display video stream 18 provided by the video cable splitter 15 or by screen mirroring) carries the content presented on the display device 24′ of the medical imaging device controller 10. The communication link 14 allows for screen sharing from the display device 24′ in the medical imaging device bay 3 to the display device 24 in the remote service center 4. The GUI 28′ includes one or more dialog screens, including, for example, an examination/scan selection dialog screen, a scan settings dialog screen, an acquisition monitoring dialog screen, among others. The GUI 28′ is included in the controller display video stream 18 and displayed on the remote workstation display 24 at the remote location 4.

FIG. 1 shows an illustrative local operator LO (also indicated in FIG. 2), and an illustrative remote expert RE. However, the ROCC optionally provides a staff of remote experts who are available to assist local operators LO at different hospitals, radiology labs, or the like. The ROCC may be housed in a single physical location or may be geographically distributed. The server computer 14s is operatively connected with one or more non-transitory storage media 26s. The non-transitory storage media 26s may, by way of nonlimiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the server computer 14s, various combinations thereof, or so forth. It is to be understood that any reference to a non-transitory medium or media 26s herein is to be broadly construed as encompassing a single medium or multiple media of the same or different types. Likewise, the server computer 14s may be embodied as a single electronic processor or as two or more electronic processors. The non-transitory storage media 26s stores instructions executable by the server computer 14s. In addition, the non-transitory computer readable medium 26s (or another database) stores data related to a set of remote experts RE and/or a set of local operators LO. The remote expert data can include, for example, skill set data, work experience data, data related to ability to work on multi-vendor modalities, data related to experience with the local operator LO and so forth.

The local operator LO may also have access to an electronic processing device computer 36 (also referred to herein as an ROCC device 36) or the like in the control room 5 that is operatively connected with the communication pathway 14. The illustrative electronic processing device computer 36 may, for example, comprise an iPad® available from Apple Corporation or an Android® tablet available from Samsung Corporation for example, but another type of electronic processing device such a notebook computer, or the controller of the imaging device itself, can also be used. The electronic processing device 36 is configured to, for example, act as the user device for the local operator LO in a video call with the remote expert RE when the natural language communication pathway 19 is operating as a video call, and could be used for various other activities such as accessing the radiology department examination schedule and/or other hospital databases such as the Electronic Medical Record of the patient undergoing the medical imaging examination.

The above-described ROCC infrastructure provides the remote expert RE with situational awareness of the imaging examination (e.g. via processed video stream 39 and controller display video stream 18) and the natural language communication pathway 19 via which the remote expert RE can provide assistance to the local operator LO. However, as recognized herein, the situational awareness of the remote expert RE could be compromised by glare, obstructions, RF screen-induced video noise, or the like that degrade the image quality of the as-acquired video stream 17. Such degradation of the video stream 17 could potentially impair the ability of the remote expert RE to maintain adequate situational awareness of the imaging examination. Embodiments disclosed herein provide the video stream processing 100 for ensuring suitable image quality of the processed video stream 39 that is viewed by the remote expert. Additionally, while the natural language communication pathway 19 comprising a telephonic or video call provides a pathway for the remote expert RE to convey advice or instructions to the local operator LO, it may be difficult to convey some types of information by these mechanisms. Hence, embodiments disclosed herein provide graphically-oriented ways for the remote expert RE to convey advice or instructions to the local operator LO. To this end, as disclosed herein the server 14s and/or remote workstation 12 implements video stream processing 100 to ensure quality of the processed video stream 39 that is sent to the remote workstation 12. The video stream processing 100 is shown in FIG. 1 as being implemented on the local operator side. This can be beneficial if, for example, the video processing 100 involves control of edge devices such as the remotely controllable lighting 8a, 8b or the PTZ 11 of the bay camera 16 (see FIG. 2). However, in other embodiments the video processing 100 may be performed at the remote site, e.g. the video stream 17 from the bay camera 16 may be transmitted to the remote site where the video stream processing 100 is applied by the server computer 14s and/or the remote workstation 12 before the processed video stream 39 is presented on the remote workstation 12.

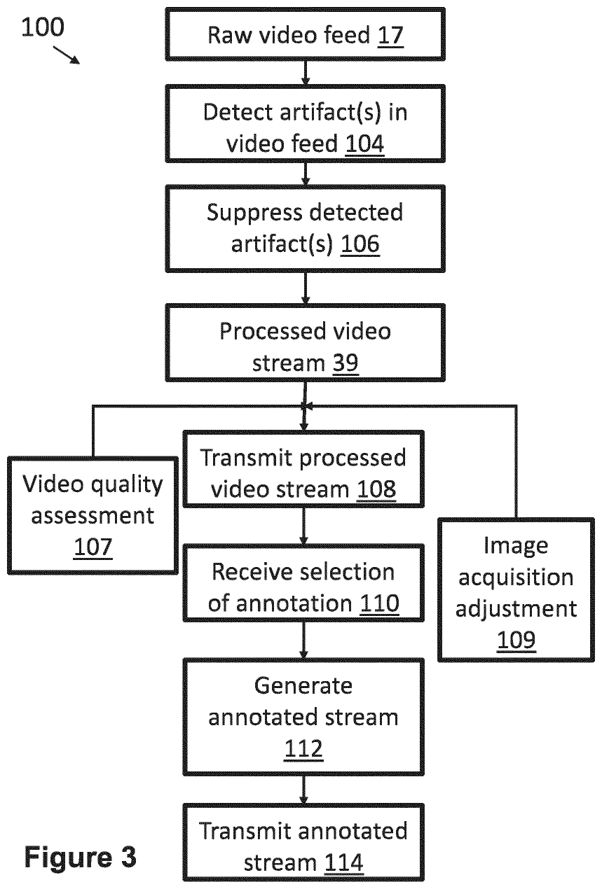

With reference now to FIG. 3, and with continuing reference to FIGS. 1 and 2, an illustrative embodiment of the video processing method 100 is diagrammatically shown as a flowchart. The input is the examination video stream 17 from the bay camera 16.

At an operation 104, one or more artifacts (e.g., glare, reflections, noise, instructions, and so forth) are detected in the received examination video stream 17. In some embodiments, the examination video stream 17 is transmitted to the server computer 14s, and the server computer 14s applies a machine-learning (ML) component 38 to the examination video stream 17 to detect one or more artifacts. Alternatively, such processing may be provided at the local operator site, e.g. by a server at the radiology department, hospital, or other medical institution hosting the medical imaging device 2.

At an operation 106, the one or more detected artifacts in the examination video stream 17 are suppressed to generate the processed video stream 39. For example, the examination video stream 17 is processed by the ML component 38 to suppress or remove the artifacts in the examination video stream 17 to generate the processed video stream 39.

In some embodiments, when the medical imaging device 2 is an MRI imaging device used in an MRI imaging examination, and the imaging bay 3 is surrounded by the RF screen/shield. The examination video stream 17 is received from the bay camera 16 viewing the MRI examination through the RF screen. The detecting operation 104 includes detecting an artifact caused by the RF screen 7 by, for example, performing an edge detection process on the examination video stream 17 to detect edge artifacts caused by the RF screen. The suppressing operation 106 includes suppressing the artifact caused by the RF screen by, for example, performing an image inpainting process on the examination video stream 17 to suppress or remove the artifact caused by the RF screen in order to produce the processed video stream 39.

In another embodiment, the detecting operation 104 includes performing an object recognition on the examination video stream 17 to identify one or more objects in the examination video stream 17, and the suppressing operation 106 includes obfuscating the identified one or more objects in the examination video stream 17 to generate the processed video stream 39.

At an operation 108, the processed video stream 39 is transmitted from the server computer 14s to the remote electronic processing device 12 for display on the display device 24 for viewing by the remote expert RE. In some embodiments, the processed video stream 39 comprises a plurality of processed video streams 39 (generated from a corresponding number of examination video streams 17). In such embodiments, at an operation 107 performed prior to the transmitting operation 108, a video quality assessment is performed on each of the processed video streams 39 to select one of the processed video streams 39 having an optimal quality. The selected processed video stream 39 is then transmitted to the remote electronic processing device 12. In some examples, after the video quality assessment operation 107 is performed, one or more parameters of the suppressing operation 106 can be adjusted based on results of the video quality assessment on the processed video streams 39.

In some embodiments, an image acquisition adjustment operation 109 is performed prior to, during, or after the transmitting operation 108. In one example, the image acquisition adjustment operation 109 includes automatically adjusting a field of view of the bay camera 16 used to acquire the examination video stream 17 in order to reduce the one or more detected artifacts in the examination video stream 17. In another example, the image acquisition adjustment operation 109 includes automatically adjusting lighting 8a of the medical imaging device bay 3 and/or lighting 8b of the control room 5 to reduce the one or more detected artifacts in the examination video stream 17. The lighting adjustment may also have some granularity, e.g. only a portion of the lights 8a in the imaging bay 3 may be dimmed (optionally up to being fully dimmed, i.e. turned off completely), and/or only a portion of the lights 8b in the adjoining control room 5 may be dimmed (optionally up to being fully dimmed, i.e. turned off completely).

In some embodiments, the natural language pathway 19 between the local operator LO and the remote excerpt RE can be established. In another example, a display of the controller 10 can be mirrored on the remote electronic processing device 12.

The foregoing operations operate to improve the video quality of the processed video stream 39. Additionally or alternatively, the video processing 100 may provide a mechanism for the remote expert RE to provide graphical advice or instructions to the local operator LO. At an operation 110, to this end once the processed video stream 39 is received at the remote electronic processing device 12, a palette of annotations 40 (stored in the server computer 14s) is displayed on the GUI 28 of the display device 24 along with the processed video stream 39. The remote expert RE then provides, via the user input device(s) 22, one or more inputs of at least one of the annotations 40 on a base image in the processed video stream 39. For example, the one or more inputs can be indicative of a selection of one of the annotations 40 from the palette of annotations 40. In some embodiments, a rendering of the imaging bay 3 can be generated and added to the base image of the processed video stream 39. In another example, a list of items to be annotated can be displayed on the GUI 28. In another example, the remote expert RE can provide an input indicative of a movement of the annotation(s) 40 on the base image in the processed video stream 39.

At an operation 112, the annotation(s) selected by the remote expert RE can be superimposed on the processed video stream 39 to generate an annotated video stream 41. In some examples, one or more of the annotations 40 can be updated in the palette of annotations 40 to indicate a status of a device or component represented by the annotation(s) 40. In another example, the list of items to be annotated can be updated when an item on the list is added to the video stream via the annotation(s) 40.

At an operation 114, the annotated video stream 41 is transmitted to the ROCC device 36 for viewing on the display device 37 by the local operator LO.

### EXAMPLE

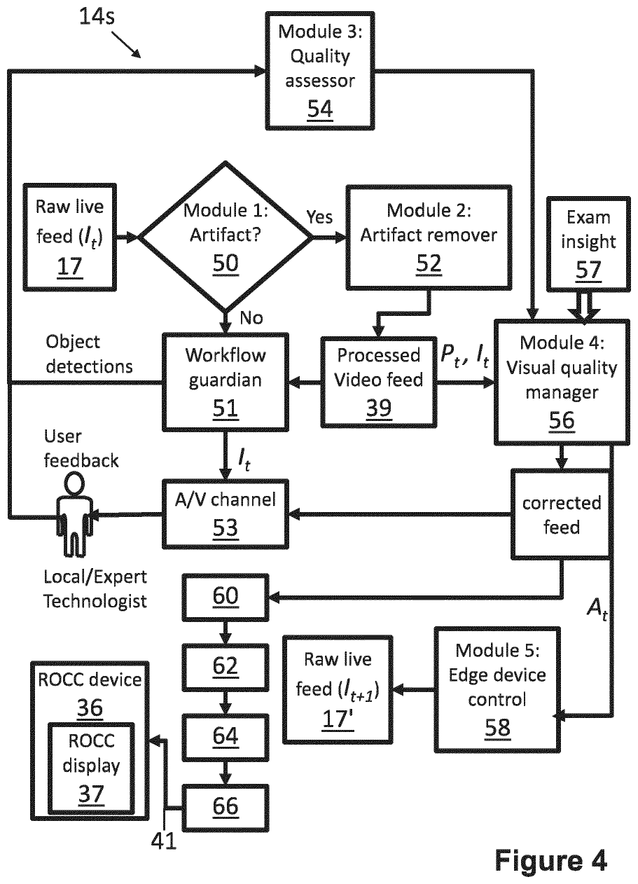

With reference to FIG. 4, a further embodiment of the ROCC system 1 is described. The server computer 14s includes one or more modules to implement the method 100. For example, as shown in FIG. 4, a first module 50 is configured to detect artifacts in the raw live examination video stream 17 (i.e., perform the detecting operation 104). This first module 50 captures an image (Iₜ) from the examination video stream 17 and it attempts to detect the artifacts in Iₜ. Artifacts such as noise are detected either using state-of-the-art algorithms or comparing Iₜ with a (stack of) template image(s) that depict(s) the usual state of the medical imaging device bay 3. Since there is a glass or plexiglass other transparent material (i.e. window 6) in the view of the camera 16, multiple artifacts can appear in an image which requires different artifact detection mechanisms for a more robust setup. For detection and localization of glare, a deep-learning approach can be based on a binary segmentation convolutional neural network model 38 (see, e.g., Chen, Y., Liu, F. and Pei, K., 2021. Self-supervised Sun Glare Detection CNN for Self-aware Autonomous Driving. Machine Learning for Autonomous Driving Workshop at the 35th Conference on Neural Information Processing Systems). The camera defocus detection problem, which especially presents a problem when there is an RF shielding mesh on glass, can be tackled by either a deep defocus blurriness detection algorithm (see, e.g., Cun, X., and Pun, C., 2020. Defocus Blur Detection via Depth Distillation. ECCV) or observation of a sharpness loss in the current frame. In order to eliminate multiple artifacts such as reflection, shadow etc. at once, a unified deep learning model for deep superimposed image decomposition can be utilized (see, e.g., Zou, Z., Lei, S., Shi, T., Shi, Z., and Ye, J., 2020. Deep Adversarial Decomposition: A Unified Framework for Separating Superimposed Images (CVPR). With an adversarial training methodology, such linear and non-linear blendings can be targeted by one model. As another perspective a more traditional method can be followed by using a template image for reference during comparison. If this module does not detect any artifacts, then the image Iₜ is directly relayed to an Audio/Video Channel 51 and a workflow guardian 53 on ROCC system 1. Otherwise, the image Iₜ is fed to a second module. To further improve the computational efficiency of the first module 50, it can be modified to select key frames from the examination video stream 17.

A second module 52 is configured to process the examination video stream 17 to remove the detected artifacts (i.e., perform the suppression operation 106). The second module 52 is configured to predominantly uses computer-vision and deep learning-based techniques to detect and correct image artifacts such as glare, noise, and reflections and sends the associated regions/contours to an interfacing module (described in more detail below).

In a first example, the second module 52 is programmed to denoise images in the examination video stream 17. Since MRI imaging bays typically have RF shield 7 on the window 6, it might reduce the video quality which would lead to performance degradation on the deep learning-based techniques and make it difficult for the remote expert to observe the imaging bay 3 in the video stream 17 acquired by the bay camera 16 viewing the imaging bay 3 through the window 6 with the RF shield 7. This degradation can be reduced by further processing of the examination video stream 17 by cleaning the image to remove noise introduced by the RF screen 7. This can be done via morphological operations and classical image processing techniques. By using Laplacian Gaussian Filter for vertical and horizontal axes, it is possible to detect the rapid changes in the image. Notably, as the RF screen 7 typically has defined directions for the metal fibers or the like that make up the mesh of the RF screen 7, it may be expected that the image noise in the video 17 should have a spatial orientation corresponding to the physical orientation of these mesh fibers, and so the Laplacian Gaussian Filter is expected to be effective for this noise suppression. Following this operation with binarization and image inpainting (via dilation), a cleaner version of an image can be obtained.

In a second example, the second module 52 is programmed to remove reflections and/or glares in the examination video stream 17. For the sake of simplicity and minimal user input during the reflection removal, single image-based reflection removal methods are used. The examination video stream 17 is a time-series of images, and each image (denoted by Iₜ) is a blending of background and reflection layers according to Iₜ=Bₜ+Rₜ where Bₜ is the background layer and Rₜ is the reflection layer.

To separate an image in the examination video stream 17 into these two layers Bₜ and Rₜ, multiple objective functions are implemented in the deep learning model 38 which also ensures that the semantic understanding plays an important role in the separation process (see, e.g., X. Zhang, R. Ng, and Q. Chen, 2018. Single image reflection separation with perceptual losses. IEEE/CVF Conference on Computer Vision and Pattern Recognition.) Another suitable approach is creating probabilistic reflection confidence maps by introducing a reflection detection via Laplacian features component to the system (see, e.g., Z. Dong, K. Xu, Y. Yang, H. Bao, W. Xu, and R. W. H. Lau, “Location-aware single image reflection removal”, IEEE/CVF International Conference on Computer Vision (ICCV)) (2021).

In the cases where the user feedback does not introduce too much additional work to the system, the second module 52 can gather feedback from both the local operator LO and the remote expert RE while correcting the images. In the background layer Bₜ, static objects such as scanner and table base need to exist. Reflections can take many forms and they can overlap with objects in the background. To address this classification problem, techniques based on motion cues and temporal information can be implemented. In addition to this, inputs can be collected from both the local operator LO and the remote expert RE to differentiate the background and reflection areas. A user assisted reflection removal can also be implemented (see, e.g., Ahmed, A., Kim, S., Elgharib, M. and Hefeeda, M., July 2021, “User-assisted video reflection removal”, in Proceedings of the 12th ACM Multimedia Systems Conference (pp. 122-131)).

Based on the detections and user feedback, the current image is processed to correct/remove the detected artifacts according to Pₜ=g(Iₜ). Notably, it is also observed that the reflection removal algorithms tend to remove glare artifacts produced by strong light sources (e.g., bay and/or control room lights 8a and/or 8b) from the images which helps to improve the video quality.

The output of the second module 52 is the processed video stream 39. An optional third module 54 is programmed to perform a quality assessment on the examination video stream 17 and processed video stream 39. With the use of a video stream quality assessment process (i.e., the operation 107) on both the processed video stream 39 (Pₜ) and examination video stream 17 (Iₜ), the effect of correction techniques from the second module 52 can be demonstrated. This assessment can be done via either deep learning or feature-based techniques. For the deep learning approach, a Neural Image Assessment method can be used where there are two models to assess the image quality in different aspects (see, e.g., Talebi, H. and Milanfar, P., 2018. NIMA: Neural Image Assessment. IEEE Transactions on Image Processing, 27(8), pp. 3998-4011). The first model is focused on the aesthetics quality of the image whereas the other model checks the technical quality. The aesthetics assessment data used for the training is the scores that the professional photographers give to the images and the technical assessment data is based on contrast, blurriness, ghosting etc. Since this method is a learning-based approach, as additional data arrives to be presented during training, the model can be adapted to our case better. This data can be the user feedback derived from local/expert technologist. For instance, users provide their scores for the current raw and processed frames where the score is lower if there is low visibility from glare or reflections and obstructions. This feedback could also include context-dependent feedback for specific regions of the image, e.g. during patient table adjustment the quality of the patient table controls and patient “contour” is most important. For the feature-based technique, a no-reference method that relies on the luminance coefficients can be used (see, e.g., Yong, C., Hao, F. and Huanlin, L., 2018. No-Reference Image Quality Assessment and Application Based on Spatial Domain Coding. IEEE Access, 6, pp.60456-60466.). These coefficients are being used for the quantification of naturalness of an image which might be highly affected by the distortions. The video assessment operation 107 can be augmented, for example, by an Improvement in detected objects by the workflow guardian on Pₜ, or applying the video stream quality assessment method on a specific region of interest in the raw or processed image. This region can be derived from several techniques including acquisition workflow context. For example, when IV related issues pop-up, IV screen is selected as the specific region of interest.

A visual quality manager (i.e., fourth) module 56 is configured to broadcast the processed video stream 39 while avoiding effects such as flickering, low resolution, which could be introduced by the processing. The visual quality manager module 56 dynamically manages the timing of image corrections in the second module 52 and control of the edge devices in a fifth module 58 so that quality of the processed video stream 39 is maintained.

In addition, the visual quality manager module 56 is configured to interact with the local operator LO and the remote expert RE, for example by alerting the local operator LO if there are unintended obstructions and noises such as IV stand in the camera's field of view, MR mesh is in the focus, or a decrease in video stream quality, or allowing the local operator LO and the remote expert RE to provide inputs such as marking reflections in the current image or regions of interest.

In addition, the visual quality manager module 56 is configured to derive exam context from multiple sources within the ROCC system 1, such as for example, providing an exam insight 57 on the ROCC system 1 to detect console actions such as scanning in progress, new patient registration etc. The workflow guardian 51 is programmed to detect workflow steps such as patient is inside the bore, patient entered the imaging bay 3, and so forth. The ROCC system 1 can identify exams that need to preserve patient's privacy during the scanning (i.e., breast screening).

Based on the exam context 57 identified, the visual quality manager module 56 identifies the appropriate set of actions (Aₜ) for each edge device and passes them to a fifth module 58 to perform the adjustment operation 109 (e.g., trigger smart LED lights 8b in the control room 5 to dim when patient is inside the scanner bore and scanning is in progress so that the visibility of the imaging bay is high, pull down the remote blinds 9 when the patient is inside the imaging bay 3 and his/her exam needs to preserve patient privacy. It also turns off the Workflow Guardian 51 and stops updating the ROCC system 1, using the PTZ (Pan Tilt and Zoom) 11 features of the camera 16 to change the field of view so that the glare from the ceiling mounted lights can be avoided or reduced. Alternatively, if multiple camera streams with different views are available, the module could switch between or mutually augment them; and so forth).

The edge device control module(s) 58 can be operated independently, but in some embodiments, adjustments are made to operate them in a way that avoids an abrupt change to the frames in the video stream 17. For example, if there are multiple smart LEDs in the control room 5, dimming a subset of smart LEDs in a sequential manner could be an appropriate choice to control the brightness in the image rather than dimming all of them at once. Such actions on a given edge device are denoted by aₜ and the collection of such actions are denoted by Aₜ. Iₜ₊₁=f(Iₜ, Aₜ)Iₜ The actions mentioned here can be implemented as a simple rule-based engine or can be derived by the AI model 38, trained on the acquisition workflow practiced in the imaging bay 3. Alternatively, alerts to perform the above actions could be given to the local operator, instead of fully automatic control.

The edge device control modules 58 can include the PTZ control 11 of the camera 16, the smart LEDs or other lighting 8a and/or 8b, the remote control blinds 8, and/or so forth. The edge devices can each have APIs through which they can be controlled. The module 58 houses all such APIs of the edge devices installed in the scanner/control room and the associated control parameters (provided by the visual quality manager module 56, as output actions Aₜ) are used to operate these edge devices in a manner that is aimed at improving the visual comfort of local/expert technologists.

FIG. 4 further illustrates an embodiment of the annotating operations 110, 112, 114 of FIG. 3. A sixth module 60 is configured to enable the remote expert to create a graphical version of exam setup in the imaging bay 3. The remote expert RE views the corrected video stream 39 from the imaging bay 3 shared by the local operator LO (i.e., the transmitting operation 108). This sixth module 60 provides the palette of annotations 40 that can be used for setting up the exam in the imaging bay 3. Based on the exam needs, the remote expert RE selects the annotations 40 needed from these lists and positions them appropriately (i.e., the operations 110 and 112). This graphical version of the exam setup is overlayed onto the live corrected video stream 39 so that the local operator can simultaneously see and interact with the remote expert RE. This form of visual interaction would enable them to communicate better between each other. This sixth module 60 can also be implemented in an offline version (for example, educational training purposes). In such cases, a template of the imaging bay 3 is used instead of the examination video stream 17.

In cases where the bay camera 16 supports pan, tilt and zoom features, the sixth module 60 enables the remote expert RE to extend their visual guidance. For instance, the remote expert RE can zoom into a specific region of the imaging bay 3 (i.e., a patient monitoring screen) and demonstrate how to operate certain devices in a safe way while scanning the patient. In such cases, the sixth module 60 handles auto-scaling the region of interest and allows the remote expert RE to use a free-hand drawing tool to guide the local operator LO. With the free-hand drawing tool, the remote expert RE can draw arrows pointing in a certain direction to indicate the patient or device needs to be moved in that direction, or draw markers of where the head/feet should be positioned etc.

A seventh module 62 is configured to use AI algorithms such as YOLOX to detect different objects used during an imaging exam in a given scanner. Some examples of these objects could be coils, pads, IV stand, patient monitoring systems etc. These identified objects are auto-populated (if not present in the initial list) in appropriate categories, as mentioned in the sixth module 60, for the corresponding imaging bay 3 at regular intervals. In another embodiment, the information on these objects could be derived by running OCR on particular fields within the console screen. In another embodiment the list of available devices/coils and/or the detection algorithm is prepopulated/supported by an inventory list created by the staff and/or sales information.

The objects used in image acquisition are prone to malfunctioning. The seventh module 62 allows the remote expert RE to mark a particular malfunctioning object as “not working”. Such feedback is also used to update the server computer 14s. In this way, the seventh module 62 facilitates an easy way to track and update the available inventory for each individual imaging bay 3. Depending on the site's service contract/sales situation this information can be fed back to/synchronized with a vendor's service/sales systems.

An eighth module 64 is configured to gather exam information such as exam name, objects, patient condition used in exams from multiple data sources. It uses this information along with AI algorithms such as collaborative filtering to recommend a checklist of items for both the remote expert RE and the local operator LO. Best practices in image acquisition can be introduced into such a checklist and thereby facilitate standardization of care while operating the imaging bay 3. It is also possible for the remote expert RE to interact with the proposed checklist by adding a new item, deleting one, marking the items that are completed, or ordering the items based on the priority given by the remote expert RE. These interactions can be used for the future examinations while doing checklist item recommendations and ordering of those items. A sample checklist contains patient monitors (IV stand, ECG monitors etc.), body part information, need for contrast agent, any identified implants, requirement of transport, and so on.

A ninth module 66 is configured to transmit the annotated video stream 41 back to the ROCC device 36 (i.e., the operation 114). The ninth module 66 is programmed to stream the annotated video stream 41 using tools such as ffmpeg or virtual cameras such as OBS studio. The annotated video stream 41 can be generated directly within the web interface displayed on the ROCC device 36. Since the information displayed on the camera overlay view is relevant for activities in the examination room, an alternative display is proposed: A touch screen display of the MR scanner controller 10 might be used, however, also an additional monitor (MR-safe device in case of an MR imaging room) can be placed in the exam room 3 to display either the same web interface as the ROCC device 36 on the console desk or a limited web interface containing only the live camera view with overlays. Additionally, this device can facilitate 2-way visual interactions between the local operator LO and the remote expert RE. Unlike in the current scenario where the feedback from the remote expert RE is obtained when the local operator comes back to the control room, this two-way communication allows remote expert to provide feedback to actions of the local operator LO instantly. In another embodiment, the visualization is accomplished using the already present monitor(s) via signal switching between the default video signal and the video information. This avoids the need for an additional monitor. In another embodiment, the ROCC device 36 can be mounted as close as possible to the glass window facing the imaging bay 3.

In another embodiment, the processed video stream 39 and overlays are realized in three dimensions (3D) instead of two-dimensions (2D). In this case, a 3D model of the exam room 3 is created and stored before using overlay technology. The 3D model can be created via a CAD modelling software or via a 3D scanning device, such as a 3D optical camera, a laser scanner, etc. To produce the live feed and overlays, a 3D camera 16 is observing the room, so that detected elements can be assigned to the correct 3D coordinates in the room and the model. To place overlay elements, the remote expert RE uses either a (simplified) CAD software (viewing the room model, possibly augmented with 3D camera data) or a VR setup (wearing AR/VR goggles and placing the overlay elements via gestures). The local operator LO would be able to view the overlay elements as augmented reality using AR/VR goggles, preferably goggles that do not cover the eyes completely, such as “Google glass” etc.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

In the foregoing detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials, and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms “a,” “an” and “the” are intended to include both singular and plural forms, unless the context clearly dictates otherwise. Additionally, the terms “comprises,” “comprising,” and/or similar terms specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

1. A non-transitory computer readable medium storing instructions for assisting a medical imaging examination of a patient, the the instructions, when executed by a processor, cause the processor to:
obtain a video stream of the medical imaging examination;
detect one or more artifacts in the received video stream;
suppress the one or more detected artifacts in the video stream to generate a processed video stream; and
transmit the processed video stream to a processing device for use by a remote user and displaying the processed video stream on a display device of the processing device.

2. The non-transitory computer readable medium of claim 1, wherein, to detect one or more artifacts in the obtained video stream, the instructions, when executed by the processor, further cause the processor to:
apply a machine-learning component to the obtained video stream to detect the one or more artifacts.

3. The non-transitory computer readable medium of claim 1, wherein:
the medical imaging examination is a magnetic resonance imaging examination performed in an imaging bay shielded by a radio frequency screen;
the video stream is obtained from a camera viewing the MRI examination through the RF screen; and
detection of the one or more artifacts includes detection of an artifact caused by the RF screen and the suppressing includes suppressing the artifact caused by the RF screen.

4. The non-transitory computer readable medium of claim 3, wherein, to detect the artifact caused by the RF screen, the instructions, when executed by the processor, further cause the processor to:
perform an edge detection process on the video stream to detect edge artifacts caused by the RF screen.

5. The non-transitory computer readable medium of claim 3, wherein, to suppress the artifact caused by the RF screen, the instructions, when executed by the processor, further cause the processor to:
perform an image inpainting process on the video stream to suppress or remove the artifact caused by the RF screen.

6. The non-transitory computer readable medium of claim 1, wherein the video stream comprises a plurality of video streams, and, the instructions, when executed by the processor, further cause the processor to:
perform a video quality assessment on the processed video streams to select one of the processed video streams having an optimal quality; and
transmit the selected processed video stream to the processing device.

7. The non-transitory computer readable medium any claim 1, wherein, the instructions, when executed by the processor, further cause the processor to:
perform a video quality assessment on the processed video streams to select one of the processed video streams having an optimal quality; and
adjust one or more parameters of the suppressing based on results of the video quality assessment on the processed video streams.

8. The non-transitory computer readable medium of claim 1, wherein:
detection of the one or more artifacts in the video stream includes performance of an object recognition on the video stream to identify one or more objects in the video stream; and
suppression the one or more detected artifacts to generate the processed video stream includes obfuscation of the identified one or more objects in the video stream to generate the processed video stream.

9. The non-transitory computer readable medium of claim 1, wherein, the instructions, when executed by the processor, further cause the processor to:
automatically adjust a field of view of at least one camera acquiring the video stream to reduce the one or more detected artifacts in the video stream.

10. The non-transitory computer readable medium of claim 1, wherein, the instructions, when executed by the processor, further cause the processor to:
automatically adjust lighting of a room to reduce the one or more detected artifacts in the video stream, wherein the room is an imaging bay within which the medical imaging examination is performed or a room within which a camera acquiring the video stream is located.

11. The non-transitory computer readable medium of claim 1, wherein, the instructions, when executed by the processor, further cause the processor to:
mirror a display of a controller of a medical imaging device used to perform the medical imaging examination at the processing device for use by a remote user; and
provide a natural communication pathway between a local operator performing the imaging examination and a remote user monitoring the medical examination.

12. A system for assisting a medical imaging examination of a patient, the apparatus comprising:
a remote processing device operable by a remote user and configured to monitor the medical imaging examination;
a local operator processing device operable by a local operator and configured to perform the medical imaging examination and having a display device; and
at least one processor configured to:
obtain a video stream of the medical imaging examination at the remote electronic processing device;
obtain, via at least one input provided by the remote user on the remote electronic processing device, at least one annotation on a base image in the video stream;
superimpose the at least one annotation on the video stream to generate an annotated video stream; and
transmit the annotated video stream to the local operator electronic processing device and displaying the annotated video stream on the display device of the local operator electronic processing device.

13. The system of claim 12, wherein the at least one processor is further configured to:
display, on the remote processing device, a palette of annotations;
wherein the obtaining includes obtaining, via the at least one input provided by the remote user on the remote processing device, a selection of the at least one annotation from the displayed palette of annotations.

14. The system of claim 12, wherein the at least one processor is further configured to:
generate a rendering of an imaging bay where the medical imaging examination is occurring; and
add the rendering of the imaging bay to the base image of the video stream.

15. The system of claim 14, wherein the at least one processor is further configured to:
update at least one annotation in the palette of annotations to indicate a status of a device or component represented by the at least one annotation.

16. The system of claim 12, wherein the at least one processor is further configured to:
provide, on a graphical user interface displayed on a display device of the remote processing device, a list of items to be annotated in the video stream; and
update the list when an item on the list is added to the video stream via the at least one annotation.

17. The system of claim 12, wherein the at least one processor is further configured to:
obtain at least one input indicative of a movement of the at least one annotation on the base image.

18. A method for assisting a medical imaging examination of a patient, the method comprising:
monitoring the medical imaging examination by recording a video stream of the medical imaging examination using a camera located in an adjoining room next to an imaging bay within which the medical imaging examination is performed and transmitting the video stream to an electronic processing device for use by a remote user and displaying the video stream on a display device of the electronic processing device; and
during the monitoring, detecting an artifact in the recorded video stream and automatically adjusting at least one of (i) the camera and/or (ii) lighting in the imaging bay and/or the adjoining room to suppress the detected artifact.

19. The method of claim 18, further comprising:
during the monitoring, detecting an artifact in the recorded video stream and automatically adjusting the camera to suppress the detected artifact.

20. The method of claim 18, further comprising:
during the monitoring, detecting an artifact in the recorded video stream and automatically adjusting lighting in the imaging bay and/or the adjoining room to suppress the detected artifact.
